(19) United States
(12) Patent Application Publication
(10) Pub. No.: US 2014/0372954 A1
(43) Pub. Date: Dec. 18, 2014

(54) Method of Forming a Prosthesis

(71) Applicant: Lifeart Prosthetics Inc., West St. Paul (CA)

(72) Inventor: Wendy A. Smith, West St. Paul (CA)

(21) Appl. No.: 14/373,825

(22) PCT Filed: Jan. 24, 2013
(86) PCT No.: PCT/CA2013/050047

§ 371 Date: Jul. 22, 2014

(30) Foreign Application Priority Data
Jan. 24, 2012 (CA) 2766109

(51) Int. Cl. A61F 2/12; G06F 3/0481; G06F 3/0484

(52) U.S. Cl. CPC A61F 2/12; G06F 3/04842; G06F 3/04817; A61F 2210/0076; A61F 2240/004
USPC 715/835; 29/527.1

(57) ABSTRACT

A prosthesis, for example a breast or a finger prosthesis, is formed for mating with a first body portion of a user such that the prosthesis is symmetrical with a second body portion of the user. The prosthesis is formed at a manufacturing location using castings of the first and second body portions which the user creates themselves at a user location separate from the manufacturing location using a user casting kit. The user casting kit includes casting material, skin colour samples and any additional tools required for the subsequent manufacture of a custom prosthesis without the user being required to leave the user location. Alternatively, the user can submit their own image data and use a computer based tool to make various selections subsequently used by the manufacturer to produce the prosthesis.

### FIELD OF THE INVENTION

The present invention relates to the manufacturing process of a custom prosthesis, in which the shape and colour of the prosthesis may be derived by: i) a user made casting and colour selections using a manufacturer supplied kit, or alternatively, ii) user captured image data and colour selections using a manufacturer supplied computer based tool.

### BACKGROUND

Reconstructive surgery is known to be performed subsequent to a mastectomy in some instance to improve body symmetry to the patient; however, this option is not available to many patients for various reasons. In this instance, an external prosthesis is commonly used to provide body symmetry to the patient. External prostheses which are available typically include either: i) mass produced prostheses; or ii) custom prostheses. Mass produced prosthesis are generally not well fitted to the user and thus are uncomfortable to wear for many users. Custom prostheses are also disadvantageous in many instances as conventional techniques for producing custom prostheses are costly, have limited availability, and require considerable interaction between the user and the manufacturer in order to be customized to the user.

### SUMMARY OF THE INVENTION

According to one aspect of the invention there is provided a method of forming a prosthesis for mating with a first body portion of a user such that the prosthesis is symmetrical with a second body portion of the user, the method comprising:

delivering a user kit to the user at a user location in which the user kit comprises a casting material;

providing a colour selection tool to the user comprising colour sample selections;

instructing the user to apply the casting material to both the first body portion and the second body portion to form respective casting portions;

instructing the user to make a colour sample selection using the colour selection tool;

receiving the casting portions delivered from the user at the user location to a manufacturing location separate from the user location;

receiving the colour sample selection selected by the user;

molding a replica of the first body portion and a replica of the second body portion of the user using the casting portions at the manufacturing location;

forming a body of the prosthesis at the manufacturing location such that the body of the prosthesis is symmetrical with the replica of the second body portion when mated with the replica of the first body portion; and

colouring the prosthesis according to the colour sample selection selected by the user.

The user casting kit effectively enables multiple users at different respective user locations to each be provided a prosthesis which is customized to them, but which is manufactured at a common manufacturing location by using the castings formed by the users not otherwise possible without the user casting kit. Accordingly a custom prosthesis can be made available to a larger number of people at a more reasonable cost than otherwise possible.

In the illustrated embodiments the prosthesis comprises a breast prosthesis such that the first body portion comprises a chest area of the user and the second body portion comprises a breast of the user. Alternatively, the prosthesis may comprise a finger prosthesis or a prosthesis for any other body part which could be fashioned from a corresponding symmetrical body part.

In the illustrated embodiments, the prosthesis comprises a breast prosthesis such that the first body portion comprises a chest area of the user and the second body portion comprises a breast of the user.

In this instance the method may further include instructing the user to fit a bra over the casting material as the casting material sets, receiving said bra with the casting portions and colour sample selection delivered from the user, and shaping a body of the prosthesis such that the body of the prosthesis fits within said bra.

Furthermore areola size samples may be provided in the kit such that the method includes instructing the user to make an areola size sample selection from the areola size samples, and colouring the prosthesis according to the areola size sample selection made by the user.

The method may further include providing tattoo selections to the user, instructing the user to make a tattoo selection from the tattoo selections, and colouring the prosthesis to include a tattoo according to the tattoo selection made by the user.

In one embodiment, the colour selection tool comprises a plurality of colour samples included in the user kit delivered to the user, and the colour sample selection comprises a selected colour sample from the user kit which is delivered from the user to the manufacturing location together with the casting portions.

Alternatively the colour selection tool comprises a computer based tool arranged to display the colour sample selections on a computer device. In this instance the colour sample selection selected by the user is delivered from the user to the manufacturing location in an electronic manner.

The computer based tool may be a web-based tool arranged to display the colour sample selections on a website and receive the colour sample selection from the user through the website.

Alternatively the computer based tool may be an application which is executable on a portable electronic device to display the colour sample selections on a display of the portable electronic device and transmit the colour sample selections to the manufacturing location over the internet.

In either instance, the computer based tool is preferably arranged to display a graphical representation of the prosthesis to the user and to vary the graphical representation of the prosthesis according to the colour sample selection made by the user, a size selection made by the user, a shape selection made by the user, an areola size selection made by the user, and an areola colour selection made by the user.

The prosthesis may be formed by i) selecting a foam core from a plurality of pre-manufactured foam cores having designated sizes and shapes such that the selected foam core corresponds to the replica of the second body portion; ii) shaping the selected foam core according to the replica of the first body portion; and iii) applying skin layers to the selected foam core according to the colour sample selection.

Preferably each of the designated sizes and shapes of the plurality of pre-manufactured foam cores is a composite average of a plurality of actual castings of second body portions of prior users.

In some instances only an inner side of the foam core is required to be shaped such that the inner side is arranged to mate with the first body portion. In other instances however, the outer side of the pre-manufactured foam core is also shaped such that the outer side is arranged to be symmetrical with the replica of the second body portion.

Alternatively, an inner side of the foam core may include a hollow portion and the method includes providing a plurality of interchangeable backing members arranged to be selectively retained within the hollow portion such that the inner side is arranged for adjustably mating with the first body portion of the user.

According to a second aspect of the present invention there is provided a method of forming a prosthesis for mating with a first body portion of a user such that the prosthesis is symmetrical with a second body portion of the user, the method comprising:

receiving a user kit at a user location in which the user kit comprises a casting material and colour samples;

applying the casting material to both the first body portion and the second body portion to form respective casting portions;

selecting a skin colour selection from the colour samples corresponding to skin tones of the user; and

delivering the casting and skin color selection from the user location to a manufacturing location separate from the user location such that a replica of the first body portion and a replica of the second body portion can be molded at the manufacturing location and such that a body of the prosthesis can be shaped at the manufacturing location to mate with the replica of the first body portion and be symmetrical with the replica of the second body portion with colouring applied according to the colour sample selection made by the user.

When the method includes fitting a bra over the casting material as the casting material cures, preferably the bra is delivered with the casting portions and colour sample selection from the user location to the manufacturing location for use in shaping a body of the prosthesis such that the body of the prosthesis fits within said bra. Preferably a waterproof sheet is positioned between the bra and casting material during the casting process.

When the method includes the user making a first colour sample selection relating to skin colour and a second colour sample selection relating to areola colour, preferably the first and second colour samples are delivered from the user location to the manufacturing location for use in colouring the prosthesis according to the first and second colour sample selections.

Similarly, when the method includes the user making an areola size sample selection from the areola size samples and/or making a tattoo selection from the tattoo samples of the kit, the areola size sample selection is preferably delivered from the user location to the manufacturing location for use in colouring the prosthesis according to the areola size sample selection made by the user and according to the tattoo sample selection made by the user.

In the preferred embodiment the colour samples each comprise a silicon member including a colour layer and a transparent layer overtop of the colour layer in which the method includes delivering the silicon member of the corresponding skin colour selection with the casting from the user location to the manufacturing location.

Preferably the kit also includes a release agent which is applied to the first and second body portions of the user prior to applying the casting material to the first and second body portions.

In some embodiments, the kit may include a camera. In this instance the method includes the user taking a photo of the second body portion and delivering the photo with the casting portions from the user location to the manufacturing location.

In the preferred embodiment, the prosthesis comprises a breast prosthesis such that the first body portion comprises a chest area of the user and the second body portion comprises a breast of the user, wherein the kit further comprises a release agent, return packaging, casting material, plastic sheeting, a measuring tape, areola size samples, skin colour samples, and areola colour samples in which the colour samples each comprise a silicon member including a colour layer and a transparent layer overtop of the colour layer. In this instance the method further includes the steps of:

i) selecting a first colour sample relating to skin colour of the user;

ii) selecting a second colour sample relating to areola colour of the user;

iii) selecting an areola size sample relating to areola size of the user;

iv) measuring about a torso of the user using the measuring tape;

v) applying the release agent to the first and second body portions of the user prior to applying the casting material to the first and second body portions;

vi) fitting a bra over the casting material as the casting material cures;

vii) positioning a waterproof sheet between the bra and casting material; and

viii) delivering said bra, the silicon members of the selected first and second colour samples, the areola size selection, and the measurement of the torso with the casting portions from the user location to the manufacturing location for use in shaping a body of the prosthesis such that the body of the prosthesis fits within said bra and for use in colouring the prosthesis according to the first and second colour samples and the areola size sample.

According to another aspect of the present invention there is provided a method of forming a prosthesis for mating with a first body portion of a user such that the prosthesis is symmetrical with a second body portion of the user, the method comprising:

providing a computer based tool to the user for use at a user location in which the computer based tool is arranged to receive image data of the first body portion and the second body portion of the user and in which the computer based tool is arranged to display a plurality of colour sample selections on a computer device to the user;

producing a computer model of the prosthesis using the image data of the first body portion and the second body portion;

receiving at the manufacturing location the colour sample selection selected by the user;

forming a body of the prosthesis at the manufacturing location according to the computer model such that the body of the prosthesis is arranged to be symmetrical with the second body portion when mated with the replica of the first body portion;

colouring the body of the prosthesis according to the colour sample selection selected by the user.

The computer based tool may be i) a web-based tool arranged to display the colour sample selections on a website and receive the colour sample selection and the image data from the user through the website, or ii) an application which is executable on a portable electronic device to display the colour sample selections on a display of the portable electronic device, to receive the image data from the user, and to transmit the colour sample selections and the image data to the manufacturing location over the Internet.

The computer based tool may be arranged to display a graphical representation of the prosthesis to the user and vary the graphical representation of the prosthesis displayed to the user according to the image data.

According to a further aspect of the present invention there is provided a method of forming a prosthesis of a respective body portion of a user, the method comprising:

providing a computer based tool to the user which is executable on a computer device for use at a user location in which the computer based tool is arranged to:
i) display a plurality of size selections to the user on the computer device;
ii) display a plurality of colour selections to the user on the computer device;
iii) generate a computer model of the prosthesis according to at least one selection made by the user; and
iv) display the computer model to the to the user on the computer device;

receiving at the manufacturing location a size selection and a colour selection selected by the user using the computer based tool;

forming a prosthesis at the manufacturing location according to the size selection and the colour selection.

The computer based tool may be i) a web-based tool arranged to display the size and colour selections on a website and receive the selections from the user through the website, or ii) an application which is executable on a portable electronic device to display the colour and size selections on a display of the portable electronic device, and to transmit the selections to the manufacturing location over the internet.

The prosthesis in this instance is preferably formed by: i) selecting a foam core from a plurality of pre-manufactured foam cores having designated sizes and shapes such that the selected foam core corresponds to the size selection made by the user using the computer based tool; and ii) applying skin layers to the selected foam core according to the colour selection. Preferably each of the designated sizes and shapes of the plurality of pre-manufactured foam cores is a composite average of a plurality of actual castings of respective body portions of prior users.

In this instance, the inner side of the foam core arranged to mate with the user preferably includes a hollow portion and a plurality of interchangeable backing members arranged to be selectively retained within the hollow portion such that the inner side is arranged for adjustably mating with the user.

Various embodiments of the invention will now be described in conjunction with the accompanying drawings in which:

### BRIEF DESCRIPTION OF THE DRAWINGS

FIG. 1 is a schematic representation of the components of the user casting kit for use according to a first embodiment of the present invention;

FIG. 2A illustrates the step of application of the release agent in the casting process according to the first embodiment of FIG. 1;

FIG. 2B illustrates the step of application of a first strip of casting material in the casting process according to the first embodiment of FIG. 1;

FIG. 2C illustrates the step of application of all of the casting material in the casting process according to the first embodiment of FIG. 1;

FIG. 2D illustrates the step of application of a waterproof sheet and bra overtop of the casting material in the casting process according to the first embodiment of FIG. 1;

FIG. 2E illustrates the step of removal of the completed casting from the user in the casting process according to the first embodiment of FIG. 1;

FIG. 3 is a flow chart representing the method of forming a prosthesis according to the first embodiment of FIG. 1;

FIG. 4 is a flow chart representing the method of forming a prosthesis according to a second embodiment;

FIG. 5 is a flow chart representing the method of forming a prosthesis according to a third embodiment;

FIG. 6 is a flow chart representing the method of forming a prosthesis according to a fourth embodiment;

FIG. 7 is a flow chart representing the method of forming a prosthesis according to a fifth embodiment;

FIG. 8 is a flow chart representing the method of forming a prosthesis according to a sixth embodiment; and

FIG. 9 is an exemplary screen shot of the computer based tool for use with various embodiments of the invention.

FIG. 10 is an exemplary prosthesis according to some embodiments of the present invention.

In the drawings like characters of reference indicate corresponding parts in the different figures.

### DETAILED DESCRIPTION

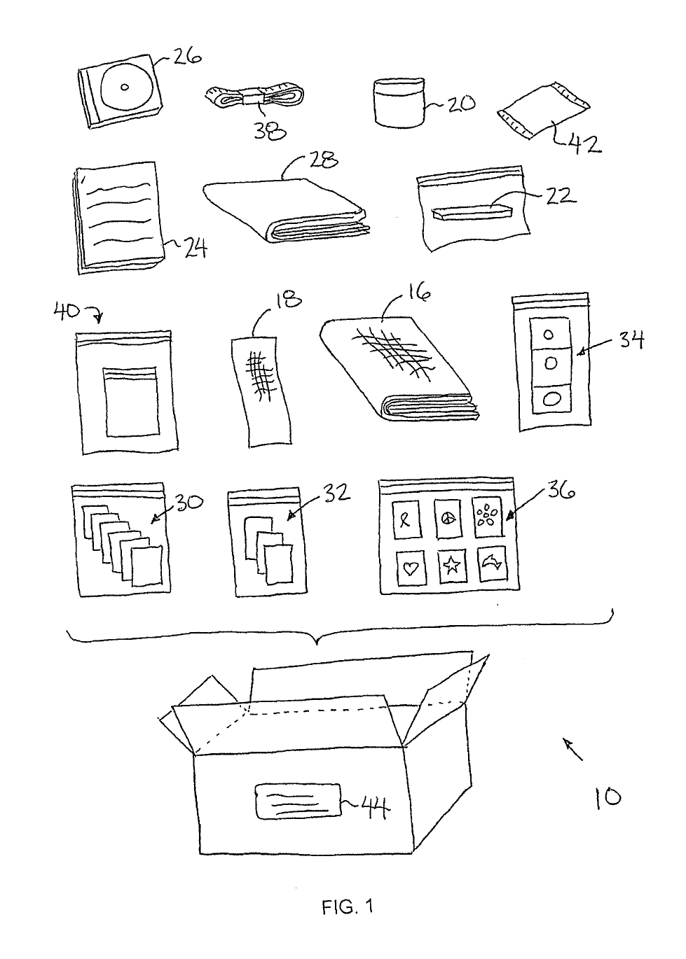

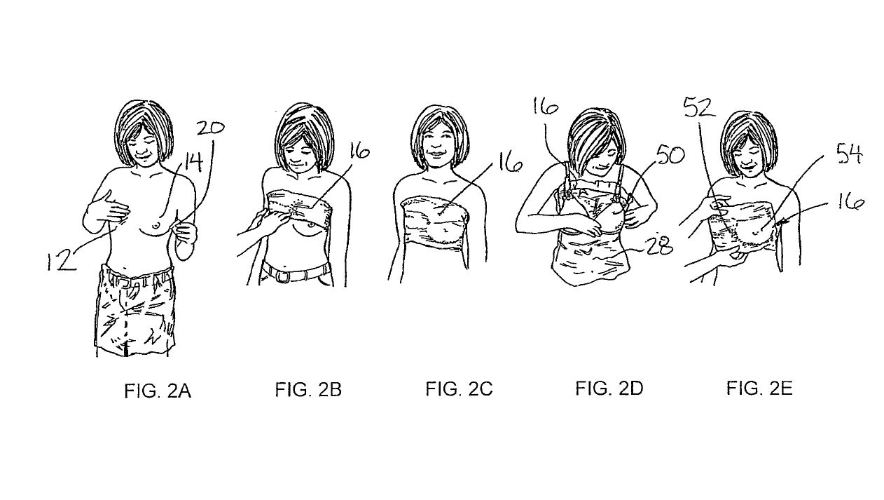

Referring initially to FIGS. 1 through 3, there is illustrated a first embodiment of a method of forming a prosthesis using a casting kit 10. The kit 10 is a tool for assisting a manufacturer to make a custom prosthesis for a user where the user produces their own castings for forming the prosthesis.

The kit is particularly suited for use with a prosthesis of the type which mates with a first body portion 12 of the user and is arranged to be symmetrical with a second body portion 16 of the user. In the illustrated embodiment, the prosthesis comprises a breast prosthesis in which the first body portion comprises a chest area to one side of the user while the second body portion comprises the breast of the user on the other side of the chest area.

In an alternative embodiment in which the prosthesis comprises a finger, the first body portion in this instance comprises a knuckle area on the hand where the prosthesis is to be worn while the second body portion comprises the corresponding finger on the other hand of the user.

The kit is packaged as a single item delivered from the manufacturer at a manufacturing location to a user at a respective user location separate from the manufacturing area. Accordingly, the kit is well suited for being delivered to the residence of the user such that the user can conduct the casting within the privacy of their own residence, while the subsequent manufacturing of the prosthesis using the castings is accomplished at a separate manufacturing location.

The kit 10 includes various components for performing the casting. A primary component of the kit are plaster bandages including main plaster bandages 16 for performing the casting as well as smaller test plaster bandages 18 for testing the casting process prior to forming the actual castings. In each instance, the plaster bandages comprise a textile material with an open weave which is coated with plaster which becomes activated when mixed with water. The kit is typically available in three different sizes in which each different kit size comprises different sizes of plaster bandages 16 for performing the casting.

The kit further includes a suitable release agent 20, for example petroleum jelly, which is applied to the skin prior to application of the casting material to protect the skin of the user and aid in releasing of the casting from the user once the casting is set.

The kit also includes a sticky-back sample adhesive in the form of an adhesive allergy test strip 22 to determine if the user is allergic to adhesive which is optionally available on the prosthesis. To perform the test, the user initially unwraps the test sample strip 22 and applies it to the skin of the user which has been cleaned and is free of any moisturizer or creams. Once the test strip has been applied to the skin in proximity to the prosthesis area, the test strip is covered with plastic wrap and worn for 24 hours to determine if there is any reaction. Upon subsequent removal of the test strip, a determination is made as to whether the user is allergic to the adhesive depending upon whether or not the skin becomes itchy or irritated. Where there is a reaction, no adhesive can be applied to the subsequently manufactured prosthesis. Where there is no reaction, the user has the option of requesting adhesive being applied to the prosthesis or not. The adhesive decision is returned from the user to the manufacturer with the castings.

Paper instructions 24 and a DVD 26 with instructions thereon are additionally provided in the kit to instruct the user through the various stages of the casting process and through various selections to be made prior to or subsequent to the casting to be returned with the casting to the manufacturer to assist in manufacturing the prosthesis.

The instructions include initially testing the casting process using the test plaster bandages 18. In this instance, the user applies an even layer of petroleum jelly to a small area in proximity to the prosthesis area. The entire skin area to be in contact with the plaster strips must be covered. To begin casting, a test strip is dipped into warm water and the excess water is gently squeezed out prior to application of the strip to the chest area. The casting material can be stretched onto the user and is smoothed out using fingertips to conform to the body contours of the user prior to wetting and applying a subsequent strip. Subsequent strips are overlapped with previous applied strips in the order of 1 inch or greater along the full length thereof. Once the user is comfortable with the process, the test strips can be removed and the casting process begun.

In the instance of a breast prosthesis, the release agent is initially applied to the entire chest area including both the first and second body portions. Prior to wetting the plaster, protective plastic sheets 28 provided in the kit are used to protect the floor and the clothing of the user by tucking the top edge of one of the sheets into the waist of the pants of the user. One of the protective sheets 28 is used as described below subsequent to the positioning of the casting material on the user.

Once a sufficient application of release agent is applied to the skin of the user, including both above and below the breast area, to both sides past the mid-line of the torso, and below the underarm area, the user can then begin applying the main plaster bandages 16. Once again, the plaster strips are wetted and then applied by stretching onto the user with each strip being smoothed to contour the body of the user prior to application of the next bandage strip with a similar overlap of approximately one inch along the full length.

The bandages are applied to fully cover the chest area including the first and second body portions across the full width of the torso from the mid-line of the torso at one side to the mid-line of the torso at the opposing side of the user. Immediately after applying the casting material and prior to curing of the casting material, one of the protective sheets 28 in the form of a waterproof plastic sheeting is applied overtop of the casting material and smoothed into place such that the user can put on their bra 50 in the usual manner. Once the bra has been properly fitted into place, the user should remain upright and standing while the plaster sets. The curing of the plaster forming the casting material initially releases heat felt by the user. Once the plaster material hardens and the heat has gone away, the cast can be carefully removed from the user and set aside. Typically, the casting material should dry for a period in the range of 48 hours to be sufficiently hardened before being returned to the manufacturing location from the user location.

In addition to returning the casting material which defines a first casting portion 52 of the first body portion and a second casting portion 54 of the second body portion respectively, the user must make several additional selections to be returned together with the casting portions. To perform these selections, the kit also includes a plurality of skin colour samples 30 and areola colour samples 32. Each of the colour samples comprises a flat silicone member formed of the same material as the prosthesis to be manufactured to ensure a true representation of the colour. More particularly, each colour sample comprises a colour layer of silicone and a transparent layer of silicone overtop of the coloured layer in accordance with the manufacturing process of the prosthesis.

In the illustrated embodiment six skin colour samples are provided corresponding to a range of different skin tones and three colour samples relating to a range of areola colours are provided. The user selects one skin colour sample and one areola colour sample which best correspond with the second body portion of the user. These selected samples are included in the package with the casting portions returned to the manufacturer. Furthermore, the kit includes three areola size samples 34 corresponding to different areola sizes for selection by the user. The areola size samples can also be returned with the casting portions if desired.

The kit further includes six tattoo samples 36 again in the form of silicone members representing the actual material of the prosthesis to be formed in which a different graphical image in the form of a tattoo is provided on each sample. The user optionally can select one of the tattoo samples to be returned together with the casting portions for being represented on the subsequent prosthesis to be manufactured.

A tape measure 38 is also enclosed within the kit to permit the user to take a circumference measurement about the torso along with any other additional measurements which may be useful for the manufacturer. These measurements are also returned with the casting to the manufacturing location.

The kit further includes sealable plastic bags 40 for use by the user. The bra which was used for fitting over the casting material is placed in one of the bags according to the instructions provided to the user for being returned to the manufacturer together with the casting portions. The other bag receives the various size and colour selections by the user. In addition to the bags 40, the casting kit further includes a desiccant material 42 included in the package delivered to the user for the user's subsequent use to be enclosed in the package with the casting material for return to the manufacturer. The user is also provided with suitable packaging including a return shipping label 44 and instructions for the user to pack the casting portions together with the various selections for return to the manufacturer.

At the manufacturing location, the manufacturer receives the casting portions together with the colour sample selections, the size selections and any other selections including tattoo selections or the adhesive selection for example. The manufacturer molds a replica of the first body portion and a replica of the second body portion using the casting portions made by the user. The replicas can be used at the manufacturing location for subsequently shaping a body of the prosthesis such that the body mates with the first body portion and is symmetrical with the second body portion when it is mated with the first body portion. The bra provided by the user can be used by the manufacturer to ensure that the prosthesis fits with the bra that was used for forming the castings.

In addition to forming the shape of the prosthesis according to the castings and the bra provided by the user, the manufacturer also colours the prosthesis according to the skin colour sample, the areola colour sample, the areola size sample, and the tattoo sample selected by the user and returned to the manufacturer. Adhesive can be applied to a portion of the prosthesis which mates with the first body portion of the user, only if the user has indicated a negative reaction to the allergy test strip and has further indicated the desire for adhesive to be provided.

In further embodiments, the kit may also be provided with a camera, for example a disposable type camera, such that the user can take a photo of the first and second body portions. The photo and the camera can be returned from the user to the manufacturing location for additional use by the manufacturer for customizing the prosthesis to the user.

According to a variant of the first embodiment of FIGS. 1 through 3, the casting material included in the kit for delivery to the user is a foam material which is easily compressible when pressed against the body of the user and then retains the impression of the corresponding body portion of the user when removed. The foam is a closed cell structured foam, for example a phenolic foam similar to the impression foam which is commercially available under the trademarked name BIO-FOAM™. In this instance, the compressed foam having the impression of the corresponding first and second body portions of the user therein is then similarly returned to the manufacturer for use in forming the prosthesis similar to the manner described above.

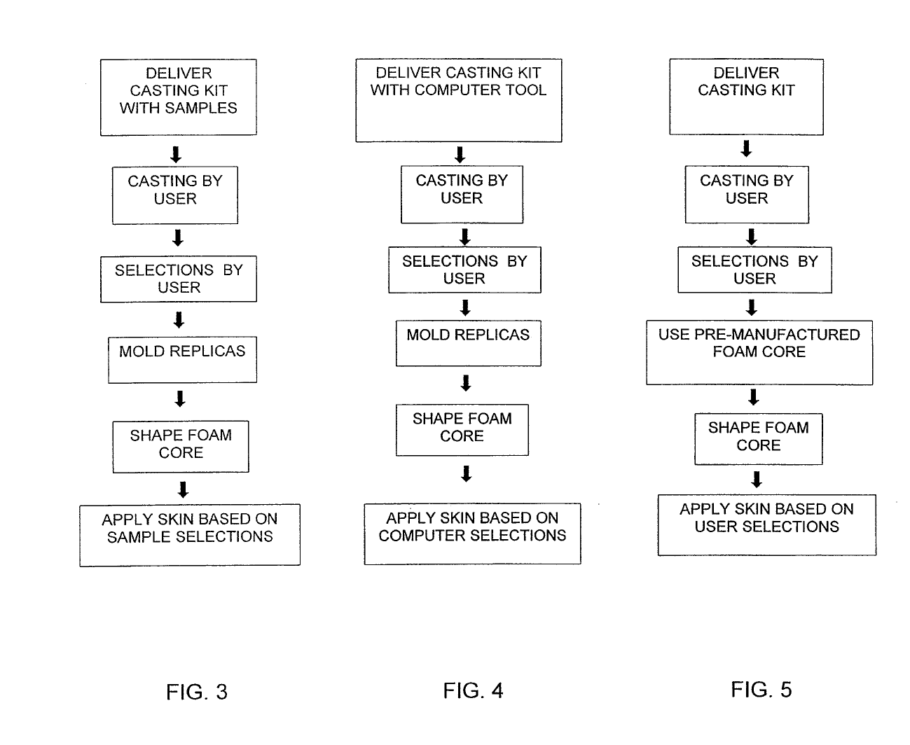

Turning now to FIG. 4, a kit is again provided as in the embodiment of FIG. 3 for performing casting in the same manner, however, the various color selections and other selections relating to the prosthesis are instead performed using a computer based tool including a color selection tool.

In one embodiment, the tool is executable on a website using a website server which displays various choices to the user as a web page and which receives selections from the user over the internet for subsequent access by the manufacturer on the website server.

Alternatively, the tool may be in the form of an application which is executable on various portable electronic devices such that the various choices and selections are displayed on a corresponding display of the electronic device. The application receives the selections and transmits the user selections over a network such as the internet to the manufacturer.

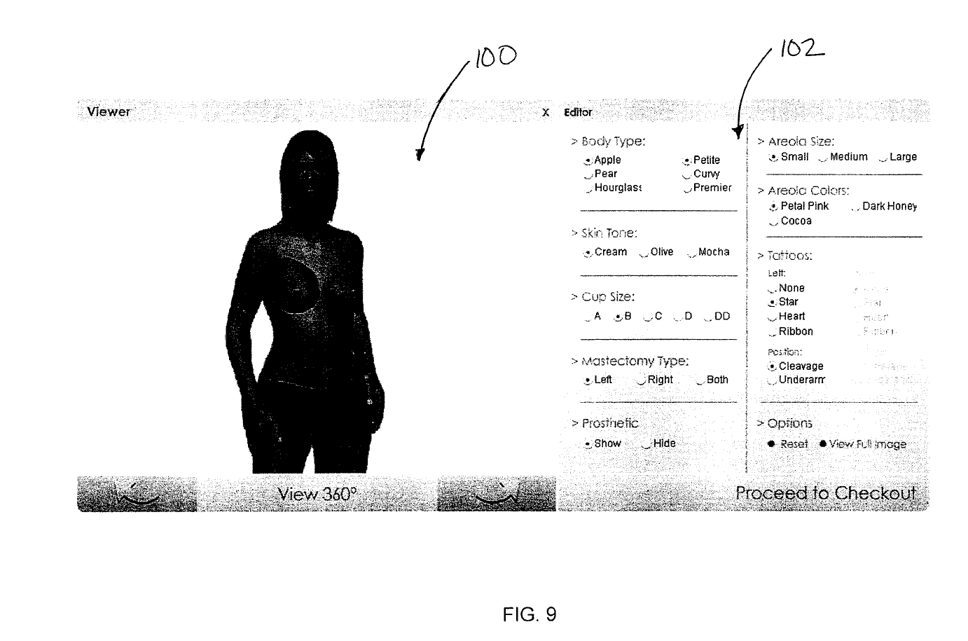

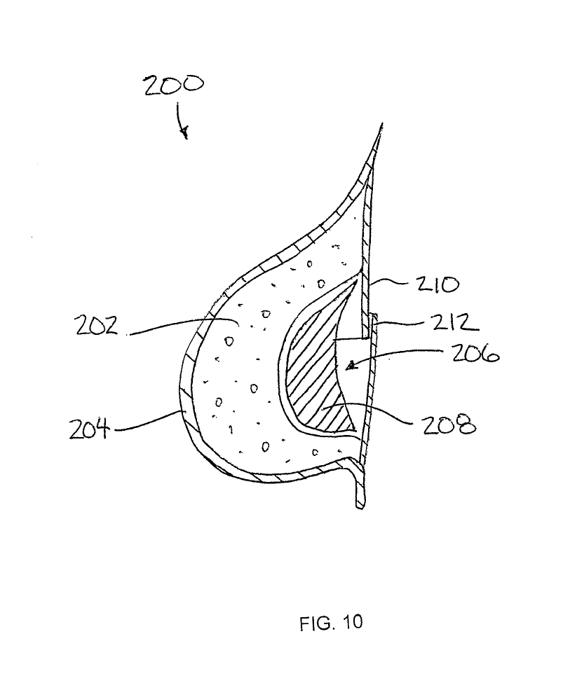

An exemplary screen shot of the computer based tool is shown in FIG. 9. The display includes a graphical portion 100 which graphically represents the various selections made by the user to graphically represent the prosthesis on the user's body. The prosthesis is represented as a three dimensional computer model which is rotatable to permit different viewing angles. Various details of the graphical model displayed on the display portion are updated as each one of various selections are made by the user.

In the example of a breast prosthesis as shown in FIG. 9, the user typically initially selects one body shape out of a plurality of different selections displayed to the user. Subsequently, the user can select a body size of a plurality of choices displayed to the user followed by skin tone and bra cup size. Each of the options similarly displays a plurality of selections to the user from which the user makes a choice and inputs the appropriate data of their selection to be stored by the computer base tool for subsequent use in manufacturing the prosthesis. When the prosthesis relates to symmetrical body parts, the user also selects the left side, the right side, or both sides to which the prosthesis relates.

At this stage a preliminary graphical model can be generated and displayed based on the selections thus far. The graphical model is then updated with each selection made of the following selections.

The additional selections include areola size, areola color, tattoo type and tattoo location if a tattoo is selected. Each of the selections is again made by presenting several options displayed to the user from which the user selects. Additional options are provided for manipulating the graphical model being displayed to permit a full body image to be displayed, or only a partial body image relating to the prosthesis area. Furthermore, the prosthesis can be hidden to view only the remaining body portion if desired. The model can also be rotated to vary the viewing angle at any time.

All of the selections are made on a separate selection area 102 of the display of the computer based tool. In the selection area 102, the current and previous selections remain displayed for subsequent review and correction by the user if desired.

When all of the selections have been made, the user completes the order and submits the selection data to the manufacturer or to a website server hosting the computer based tool. The manufacturing of the prosthesis then proceeds similarly to the previous embodiments. That is, the manufacturer initially shapes a foam core to correspond with the replicas of the first and second body portions made using the castings. Silicone layers are then applied in an uncured state to the exterior of the shaped foam core and cured in place on the foam to form skin layers which correspond to the various selections made by the user.

Turning now to the embodiment of FIG. 5, a user kit is delivered to the user similarly to the embodiments of FIGS. 3 and 4 noted above and color selections are made according to either of the embodiments of FIG. 3 or 4. The embodiment of FIG. 5 differs from the previous embodiments in that the manufacturing process starts with selecting a pre-manufactured and pre-shaped foam core blank from a limited number of prescribed shapes and sizes of blanks. The prescribed pre-shaped blanks are formed using data from prior castings or computer models from scanning of actual body parts corresponding to the prosthesis from prior prosthesis patients. After many castings or three dimensional scanned computer models of a corresponding body part have been submitted to the data base, the actual models are separated into several categories corresponding to similar shapes or sizes and the multiple actual models within each category are combined into a composite average shape and size resulting in a limited number of prescribed foam core blanks to choose from.

Subsequent manufacturing processes then permit actual castings of users which produce replicas of the first and second body portions to be compared to the prescribed blanks so that a pre-manufactured foam blank can be readily selected which most resembles the replicas of the current user for which the prosthesis is being made. Typically, the inner side corresponding to the rear side of the prosthesis to be mated against the first body portion of the user is still shaped to specifically mate with the first body portion in a custom manner while the outer side requires no or only minimal modification where it is already substantially symmetrical with the second body portion in its pre-manufactured form. The uncured silicone applied to the outer side to represent the skin layers once cured is still applied in a custom manner according to the various selections made by the user corresponding to color, areola, detail and a tattoo for example.

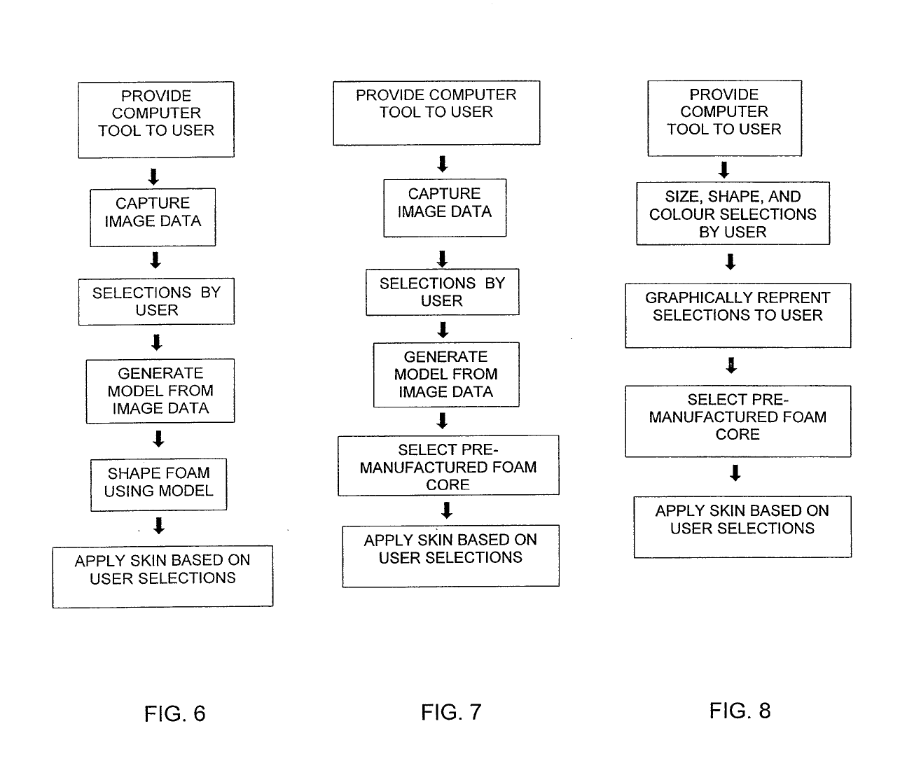

Turning now to the embodiment of FIG. 6, in this instance, the casting step of the method for forming a prosthesis is instead replaced by computer modelling of the first and second body portions using captured images of the user. In this instance, the computer based tool which is executable on a website or as an application on a portable electronic device as described above includes the additional functionality of guiding the user to capture images of themselves either as a series of static images or a video comprising a sequence of images from various changing viewing angles. The computer based tool may communicate directly with a camera already associated with the computer device upon which the computer based tool is being executed. For example, the computer based tool may retrieve images from a web cam of a computer used to view the website upon which the computer based tool is operated. Alternatively the computer based tool can be executed as an application on a portable device such as a smart phone and the tool communicated directly with the integral camera of the smart phone to capture image data.

In either instance, typically the user is initially instructed to locate reference markers on their body in proximity to the first and second body portions. Subsequently, images are captured from several different directions so that the computer based tool can combine the images by using the reference markers to identify the relationship of the various images to one another to assemble the captured images into a three dimensional computer model. Alternatively a separate computer tool may be used to capture images and generate the three dimensional computer model which is then simply submitted with the subsequent color selection made using the computer based tool as described above.

The computer application or website server may be used to assemble the computer model such that the graphical model displayed on the display portion 100 of the computer based tool is an accurate representation of the computer model assembled from the captured image data. Alternatively, the graphical model displayed on the display portion 100 may simply be an approximation based on user selections but the computer model is not actually assembled from the computer images until a subsequent step at the manufacturing location using a separate computer device.

When generating a computer model from image data, the model can be used to readily manufacture a precisely shaped foam core which can readily mate with the first body portion of the user and be symmetrical with the second body portion of the user. A suitable foam core may be formed by various rapid prototyping or 3D printing technologies for example. It is desirable that the foam core remain a light weight compressible foam. Once an accurately shaped foam core is provided, uncured silicone is applied to the outer surface to cure and form the skin layers according to the selections made using the color selection tool as described above.

Turning now to the embodiment of FIG. 7, in this instance, casting is again replaced with a computer modelling tool of the computer based tool executable on a website or as an application on a portable electronic device similar to the embodiment of FIG. 6. Color selections are also performed similarly to the previous embodiments using the computer based tool. FIG. 7 differs from the embodiment of FIG. 6 in that a less detailed computer model is required as the computer model is only used for comparison purposes to the limited number of pre-manufactured foam core blanks described above. As in previous embodiments, each pre-manufactured blank is one of a limited number of blanks corresponding to a prescribed shape and size which represents a composite average of numerous actual models of actual prior prosthetic patients to simplify the modelling and manufacturing processes. The prescribed blank which most closely corresponds to the computer model is selected and typically only the rear side is shaped to provide better mating with the first body portion of the user. The outer side may require some minor shaping or the application of silicone to form skin layers may also be used for refining the outer shape as in previous embodiments. Also as in previous embodiments, the silicone skin layers are applied according to the various color and detail selections made by the user using the computer based tool.

Turning now to the embodiment of FIG. 8, in this instance, the user only supplies information to the manufacturer using the computer based selection tool represented in FIG. 9. In this instance a separate prescribed foam core blank is designated for each of the various selection combinations of body shape, body size, bra cup size and left or right body symmetry. Based on these selections, the prosthesis 200 is initially formed by selecting a form core blank 202 at the manufacturing level to which the silicone skin layers 204 can be directly applied corresponding to the various color and detail selections entered by the user using the computer based tool. The rear or inner side of the prosthesis in this instance typically locates a hollow recessed portion 206 therein into which one of a plurality of interchangeable backing members 208 can be received and selectively retained. Various retention methods to retain a selected one of the backing members in the hollow recessed portion include: i) releasable fastening techniques for example mating hook and loop fasteners, ii) adhesive, or iii) a flexible covering 210 spanning the rear side of the prosthesis with a slotted access opening 212 therein which can be manipulated to allow insertion and removal of the interchangeable backing members therethrough. The interchangeable backing members can be of different volume, different mass, different shape, different material, or any combination thereof relative to one another. In this instance, the mating of the rear inner side to the first body portion of the user can be customized by the user subsequent to the manufacturing stages of the general shape, size and coloring of the outer side of the prosthesis to provide a near custom experience for the user with a minimum degree of customization required by the manufacturer.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A method of forming a prosthesis for mating with a first body portion of a user such that the prosthesis is symmetrical with a second body portion of the user, the method comprising:
delivering a user kit to the user at a user location in which the user kit comprises a casting material;
providing a colour selection tool to the user comprising colour sample selections;
instructing the user to apply the casting material to both the first body portion and the second body portion to form respective casting portions;
instructing the user to make a colour sample selection using the colour selection tool;
receiving the casting portions delivered from the user at the user location to a manufacturing location separate from the user location;
receiving the colour sample selection selected by the user;
molding a replica of the first body portion and a replica of the second body portion of the user using the casting portions at the manufacturing location;
forming a body of the prosthesis at the manufacturing location such that the body of the prosthesis is symmetrical with the replica of the second body portion when mated with the replica of the first body portion; and
colouring the prosthesis according to the colour sample selection selected by the user.

2. The method according to claim 1 wherein the prosthesis comprises a breast prosthesis such that the first body portion comprises a chest area of the user and the second body portion comprises a breast of the user.

3. The method according to claim 2 including providing areola size samples in the kit, instructing the user to make an areola size sample selection from the areola size samples, and colouring the prosthesis according to the areola size sample selection made by the user.

4. The method according to either one of claim 2 or 3 including instructing the user to fit a bra over the casting material as the casting material sets, receiving said bra with the casting portions and colour sample selection delivered from the user, and shaping a body of the prosthesis such that the body of the prosthesis fits within said bra.

5. The method according to any one of claims 1 through 4 including providing tattoo selections to the user, instructing the user to make a tattoo selection from the tattoo selections, and colouring the prosthesis to include a tattoo according to the tattoo selection made by the user.

6. The method according to any one of claims 1 through 5 wherein the colour selection tool comprises a plurality of colour samples included in the user kit delivered to the user, and the colour sample selection comprises a selected colour sample from the user kit which is delivered from the user to the manufacturing location together with the casting portions.

7. The method according to any one of claims 1 through 5 wherein the colour selection tool comprises a computer based tool arranged to display the colour sample selections on a computer device and wherein the method includes delivering the colour sample selection selected by the user from the user to the manufacturing location in an electronic manner.

8. The method according to claim 7 wherein the computer based tool is a web-based tool arranged to display the colour sample selections on a website and receive the colour sample selection from the user through the website.

9. The method according to claim 7 wherein the computer based tool is an application which is executable on a portable electronic device to display the colour sample selections on a display of the portable electronic device and transmit the colour sample selections to the manufacturing location over the internet.

10. The method according to any one of claims 7 through 9 wherein the computer based tool is arranged to display a graphical representation of the prosthesis to the user and the computer based tool is arranged to vary the graphical representation of the prosthesis according to the colour sample selection made by the user.

11. The method according to any one of claims 7 through 10 wherein the computer based tool is arranged to display a graphical representation of the prosthesis to the user and the computer based tool is arranged to vary the graphical representation of the prosthesis displayed to the user according to a size selection made by the user.

12. The method according to any one of claims 7 through 11 wherein the computer based tool is arranged to display a graphical representation of the prosthesis to the user and the computer based tool is arranged to vary the graphical representation of the prosthesis displayed to the user according to a shape selection made by the user.

13. The method according to any one of claims 7 through 12 wherein the prosthesis comprises a breast prosthesis, wherein the computer based tool is arranged to display a graphical representation of the prosthesis to the user and wherein the computer based tool is arranged to vary the graphical representation of the prosthesis displayed to the user according to an areola size selection made by the user.

14. The method according to any one of claims 7 through 13 wherein the prosthesis comprises a breast prosthesis, wherein the computer based tool is arranged to display a graphical representation of the prosthesis to the user and wherein the computer based tool is arranged to vary the graphical representation of the prosthesis displayed to the user according to an areola colour selection made by the user.

15. The method according to any one of claims 1 through 14 including forming the prosthesis by:
selecting a foam core from a plurality of pre-manufactured foam cores having designated sizes and shapes such that the selected foam core corresponds to the replica of the second body portion;
shaping the selected foam core according to the replica of the first body portion; and
applying skin layers to the selected foam core according to the colour sample selection.

16. The method according to claim 15 wherein each of the designated sizes and shapes of the plurality of pre-manufactured foam cores is a composite average of a plurality of actual castings of second body portions of prior users.

17. The method according to either one of claim 15 or 16 wherein only an inner side of the foam core is shaped such that the inner side is arranged to mate with the first body portion.

18. The method according to either one of claim 15 or 16 wherein an inner side of the foam core is shaped such that the inner side is arranged to mate with the replica of the first body portion and an outer side of the foam core is shaped such that the outer side is arranged to be symmetrical with the replica of the second body portion.

19. The method according to any one of claims 15 through 18 wherein an inner side of the foam core includes a hollow portion and wherein the method includes providing a plurality of interchangeable backing members arranged to be selectively retained within the hollow portion such that the inner side is arranged for adjustably mating with the first body portion of the user.

20. A method of forming a prosthesis for mating with a first body portion of a user such that the prosthesis is symmetrical with a second body portion of the user, the method comprising:
receiving a user kit at a user location in which the user kit comprises a casting material and colour samples;
applying the casting material to both the first body portion and the second body portion to form respective casting portions;
selecting a skin colour selection from the colour samples corresponding to skin tones of the user; and
delivering the casting and skin color selection from the user location to a manufacturing location separate from the user location such that a replica of the first body portion and a replica of the second body portion can be molded at the manufacturing location and such that a body of the prosthesis can be shaped at the manufacturing location to mate with the replica of the first body portion and be symmetrical with the replica of the second body portion with colouring applied according to the colour sample selection made by the user.

21. The method according to claim 20 wherein the prosthesis comprises a breast prosthesis such that the first body portion comprises a chest area of the user and the second body portion comprises a breast of the user.

22. The method according to claim 21 including fitting a bra over the casting material as the casting material cures.

23. The method according to claim 22 including delivering said bra with the casting portions and colour sample selection from the user location to the manufacturing location for use in shaping a body of the prosthesis such that the body of the prosthesis fits within said bra.

24. The method according to any one of claims 21 through 23 including positioning a waterproof sheet between the bra and casting material.

25. The method according to any one of claims 21 through 24 wherein the colour samples in the kit relate to skin colour and areola colour and wherein the method includes the user making a first colour sample selection relating to skin colour and a second colour sample selection relating to areola colour and delivering the first and second colour samples from the user location to the manufacturing location for use in colouring the prosthesis according to the first and second colour sample selections.

26. The method according to any one of claims 21 through 25 wherein the kit includes areola size samples and wherein the method includes the user making an areola size sample selection from the areola size samples and delivering the areola size sample selection from the user location to the manufacturing location for use in colouring the prosthesis according to the areola size sample selection made by the user.

27. The method according to any one of claims 20 through 26 including providing tattoo samples in the kit, instructing the user to make a tattoo selection from the tattoo samples of the kit, and colouring the prosthesis to include a tattoo according to the tattoo sample selection made by the user.

28. The method according to any one of claims 20 through 27 wherein the colour samples each comprise a silicon member including a colour layer and a transparent layer overtop of the colour layer and wherein the method includes delivering the silicon member of the corresponding skin colour selection with the casting from the user location to the manufacturing location.

29. The method according to any one of claims 20 through 28 wherein the kit includes a release agent and wherein the method includes applying the release agent to the first and second body portions of the user prior to applying the casting material to the first and second body portions.

30. The method according to any one of claims 20 through 29 wherein the kit includes an adhesive allergy test strip and wherein the method includes applying the adhesive allergy test strip to skin of the user to determine if the user has an allergic reaction, making an adhesive selection if the user does not have an allergic reaction to the adhesive allergy test strip and delivering the adhesive selection with the casting portions from the user location to the manufacturing location for use in applying an adhesive to the body of the prosthesis according to the adhesive selection.

31. The method according to any one of claims 20 through 30 wherein the kit includes a camera and wherein the method includes the user taking a photo of the second body portion and delivering the photo with the casting portions from the user location to the manufacturing location.

32. The method according to any one of claims 20 through 31 wherein the prosthesis comprises a breast prosthesis such that the first body portion comprises a chest area of the user and the second body portion comprises a breast of the user and wherein the kit further comprises a release agent, return packaging, casting material, plastic sheeting, a measuring tape, areola size samples, skin colour samples, and areola colour samples in which the colour samples each comprise a silicon member including a colour layer and a transparent layer overtop of the colour layer, the method further comprising:
selecting a first colour sample relating to skin colour of the user;
selecting a second colour sample relating to areola colour of the user;
selecting an areola size sample relating to areola size of the user;
measuring about a torso of the user using the measuring tape;
applying the release agent to the first and second body portions of the user prior to applying the casting material to the first and second body portions;
fitting a bra over the casting material as the casting material cures;
positioning a waterproof sheet between the bra and casting material; and
delivering said bra, the silicon members of the selected first and second colour samples, the areola size selection, and the measurement of the torso with the casting portions from the user location to the manufacturing location for use in shaping a body of the prosthesis such that the body of the prosthesis fits within said bra and for use in colouring the prosthesis according to the first and second colour samples and the areola size sample.

33. A method of forming a prosthesis for mating with a first body portion of a user such that the prosthesis is symmetrical with a second body portion of the user, the method comprising:
providing a computer based tool to the user for use at a user location in which the computer based tool is arranged to receive image data of the first body portion and the second body portion of the user and in which the computer based tool is arranged to display a plurality of colour sample selections on a computer device to the user;
producing a computer model of the prosthesis using the image data of the first body portion and the second body portion;
receiving at the manufacturing location the colour sample selection selected by the user;
forming a body of the prosthesis at the manufacturing location according to the computer model such that the body of the prosthesis is arranged to be symmetrical with the second body portion when mated with the replica of the first body portion;
colouring the body of the prosthesis according to the colour sample selection selected by the user.

34. The method according to claim 33 wherein the computer based tool is a web-based tool arranged to display the colour sample selections on a website and receive the colour sample selection and the image data from the user through the website.

35. The method according to claim 33 wherein the computer based tool is an application which is executable on a portable electronic device to display the colour sample selections on a display of the portable electronic device, to receive the image data from the user, and to transmit the colour sample selections and the image data to the manufacturing location over the internet.

36. The method according to any one of claims 33 through 35 wherein the computer based tool is arranged to display a graphical representation of the prosthesis to the user and the computer based tool is arranged to vary the graphical representation of the prosthesis according to the colour sample selection made by the user.

37. The method according to any one of claims 33 through 36 wherein the computer based tool is arranged to display a graphical representation of the prosthesis to the user and the computer based tool is arranged to vary the graphical representation of the prosthesis displayed to the user according to a size selection made by the user.

38. The method according to any one of claims 33 through 37 wherein the computer based tool is arranged to display a graphical representation of the prosthesis to the user and the computer based tool is arranged to vary the graphical representation of the prosthesis displayed to the user according to a shape selection made by the user.

39. The method according to any one of claims 33 through 38 wherein the prosthesis comprises a breast prosthesis, wherein the computer based tool is arranged to display a graphical representation of the prosthesis to the user and wherein the computer based tool is arranged to vary the graphical representation of the prosthesis displayed to the user according to an areola size selection made by the user.

40. The method according to any one of claims 33 through 39 wherein the prosthesis comprises a breast prosthesis, wherein the computer based tool is arranged to display a graphical representation of the prosthesis to the user and wherein the computer based tool is arranged to vary the graphical representation of the prosthesis displayed to the user according to an areola colour selection made by the user.

41. The method according to any one of claims 33 through 40 wherein the computer based tool is arranged to display a graphical representation of the prosthesis to the user and wherein the computer based tool is arranged to vary the graphical representation of the prosthesis displayed to the user according to the image data.

42. The method according to any one of claims 33 through 41 including forming the prosthesis by:
selecting a foam core from a plurality of pre-manufactured foam cores having designated sizes and shapes such that the selected foam core corresponds to an outer side of the computer model; and
applying skin layers to the selected foam core according to the colour sample selection.

43. The method according to claim 42 wherein each of the designated sizes and shapes of the plurality of pre-manufactured foam cores is a composite average of a plurality of actual castings of second body portions of prior users.

44. The method according to either one of claim 42 or 43 including shaping an inner side of the foam core such that the inner side corresponds to an inner side of the computer model and is arranged to mate with the first body portion.

45. The method according to either one of claim 42 or 43 wherein an inner side of the foam core includes a hollow portion and wherein the method includes providing a plurality of interchangeable backing members arranged to be selectively retained within the hollow portion such that the inner side is arranged for adjustably mating with the first body portion of the user.

46. The method according to any one of claims 33 through 45 wherein the prosthesis comprises a breast prosthesis such that the first body portion comprises a chest area of the user and the second body portion comprises a breast of the user.

47. A method of forming a prosthesis of a respective body portion of a user, the method comprising:
providing a computer based tool to the user which is executable on a computer device for use at a user location in which the computer based tool is arranged to:
i) display a plurality of size selections to the user on the computer device;
ii) display a plurality of colour selections to the user on the computer device;
iii) generate a computer model of the prosthesis according to at least one selection made by the user; and
iv) display the computer model to the to the user on the computer device;
receiving at the manufacturing location a size selection and a colour selection selected by the user using the computer based tool;
forming a prosthesis at the manufacturing location according to the size selection and the colour selection.

48. The method according to claim 47 wherein the computer based tool is a web-based tool arranged to display the size and colour selections on a website and receive the selections from the user through the website.

49. The method according to claim 47 wherein the computer based tool is an application which is executable on a portable electronic device to display the colour and size selections on a display of the portable electronic device, and to transmit the selections to the manufacturing location over the internet.

50. The method according to any one of claims 47 through 49 wherein the computer based tool is arranged to vary the computer model displayed to the user according to a size selection made by the user.

51. The method according to any one of claims 47 through 50 wherein the computer based tool is arranged to vary the computer model displayed to the user according to a colour selection made by the user.

52. The method according to any one of claims 47 through 51 wherein the computer based tool is arranged to display a plurality of shape selections to the user on the computer device and the computer based tool is arranged to vary the computer model displayed to the user according to a shape selection made by the user.

53. The method according to any one of claims 47 through 52 wherein the prosthesis comprises a breast prosthesis, wherein the computer based tool is arranged to display a plurality of areola colour selections to the user on the computer device, and wherein the computer based tool is arranged to vary the computer model displayed to the user according to an areola colour selection made by the user.

54. The method according to any one of claims 47 through 53 wherein the prosthesis comprises a breast prosthesis, wherein the computer based tool is arranged to display a plurality of areola size selections to the user on the computer device, and wherein the computer based tool is arranged to vary the computer model displayed to the user according to an areola size selection made by the user.

55. The method according to any one of claims 47 through 54 including forming the prosthesis by:
selecting a foam core from a plurality of pre-manufactured foam cores having designated sizes and shapes such that the selected foam core corresponds to the size selection made by the user using the computer based tool; and
applying skin layers to the selected foam core according to the colour selection.

56. The method according to claim 55 wherein each of the designated sizes and shapes of the plurality of pre-manufactured foam cores is a composite average of a plurality of actual castings of respective body portions of prior users.

57. The method according to either one of claim 55 or 56 wherein an inner side of the foam core is arranged to mate with the user and the inner side of the foam core includes a hollow portion, and wherein the method includes providing a plurality of interchangeable backing members arranged to be selectively retained within the hollow portion such that the inner side is arranged for adjustably mating with the user.

58. The method according to any one of claims 47 through 57 wherein the prosthesis comprises a breast prosthesis.
